(19) United States
(12) Patent Application Publication
(10) Pub. No.: US 2025/0268703 A1
(43) Pub. Date: Aug. 28, 2025

(54) ADJUSTABLE PATERLLAR TENDON REALIGNMENT IMPLANT

(71) Applicant: ZKR Orthopedics, Inc., Kentfield, CA (US)

(72) Inventors: Damien SHULOCK, San Francisco, CA (US); Jeffrey HALBRECHT, San Francisco, CA (US); John BARRETT, San Francisco, CA (US); Katherine J. STEPHENSON, Belmont, CA (US)

(21) Appl. No.: 19/207,860

(22) Filed: May 14, 2025

Related U.S. Application Data
(63) Continuation-in-part of application No. 17/316,233, filed on May 10, 2021, now Pat. No. 12,303,396.
(60) Provisional application No. 63/022,977, filed on May 11, 2020.

(51) Int. Cl. A61F 2/08; A61F 2/30; A61F 2/38

(52) U.S. Cl. CPC A61F 2/08; A61F 2/30749; A61F 2002/3054; A61F 2002/30624; A61F 2002/3881

(57) ABSTRACT

An orthopedic implant with an inferior portion having a tibia contact surface configured to extend over a tibia; a superior portion opposite to the inferior portion having a tendon contact surface configured to change a position of a patellar tendon by lifting or tilting the patellar tendon when the curved surface of the first portion is engaged with the tibia; a fixation mechanism adapted to attach the orthopedic implant to the tibia; and an adjustment mechanism adapted to change a distance between the tendon contact surface and the tibia contact surface. The invention also includes a method for repositioning a patellar tendon of a patient.

### CROSS REFERENCE TO RELATED APPLICATIONS

This application claims priority as a continuation-in-part of U.S. application Ser. No. 17/316,233, filed May 10, 2021 and entitled “Adjustable Patellar Tendon Realignment Implant,” which claims the benefit of priority of U.S. Provisional Application No. 63/022,977, filed May 11, 2020, which are hereby incorporated herein by reference in their entireties.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Problems of the patella-femoral joint are a common cause of knee pain. The pain may arise from issues such as poor alignment of the patella or from cartilage breakdown (chondromalacia or arthritis) behind the patella or on the opposing articular surface of the femoral groove (trochlea). Conventional surgical options for treating patella-femoral pain caused by malalignment, chondromalacia or arthritis may include realignment of the patella. For example tracking of the patella may be changed by tilting the patella or by moving the patella to one side or the other. Moving the patella forward (i.e., anteriorly) through a surgical procedure provides another conventional option for treating these conditions. This conventional technique is thought to decrease force across the patella-femoral joint and thus diminish the pain arising from excess force against a worn-out patellar or trochlear cartilage.

Although available, surgical options to realign the patella may be very invasive. For example, surgeries may involve cutting and fixating the bony attachment of the patellar tendon. In particular, conventional techniques may include detaching the patellar tendon from the tibia, then reattaching the patellar tendon at a new location to obtain the desired alignment of the patella. Such invasive surgical techniques may also result in prolonged recovery times. Consequently, an improved mechanism for treating patella-femoral joint problems such as patella-femoral pain, chondromalacia, and/or arthritis is desired.

U.S. Pat. No. 9,808,289 discloses embodiments of a patellar tendon realignment implant configured to be placed between the patellar tendon and the tibia in proximity to the patella to elevate and/or tilt the patellar tendon. Each of the implants has a fixed height and cannot be adjusted prior to, or after, implantation to meet the patient's needs.

### SUMMARY OF THE DISCLOSURE

Some activities result in higher tension or compression of the patellar tendon. For example, stair climbing and jumping increase the tension on the patellar tendon, and kneeling or sudden impacts to the knee increase patellar tendon compression. It may be desirable to temporarily change the shape of a patellar tendon realignment implant during application of a tension load on the patellar tendon (e.g., during stair climbing, jumping, etc.) or application of a compression load on the patellar tendon (e.g., during kneeling, a sudden impact to the knee, etc.).

One aspect of the invention provides an orthopedic implant with an inferior portion having a tibia contact surface configured to extend over a tibia; a superior portion opposite to the inferior portion having a tendon contact surface configured to change a position of a patellar tendon by lifting or tilting the patellar tendon when the curved surface of the first portion is engaged with the tibia; a fixation mechanism adapted to attach the orthopedic implant to the tibia; and an adjustment mechanism adapted to change a distance between the tendon contact surface and the tibia contact surface.

In some embodiments, the orthopedic implant also has a hinge or pivot connecting the superior portion to the inferior portion.

In some embodiments, the adjustment mechanism includes a piston disposed between the superior portion and the inferior portion. In some such embodiments, the adjustment mechanism includes a port fluidly connected with an interior of the piston through which fluid can be added or removed from the interior of the piston. In other such embodiments, the piston has a threaded connection between a superior piston element and an inferior piston element.

In some embodiments, the adjustment mechanism includes a rotatable cam disposed between the superior portion and the inferior portion.

In some embodiments, the adjustment mechanism includes an adjustable connection between the superior portion and the inferior portion adapted and configured to enable a user to move the superior portion along a ramp to change the distance between the tendon contact surface and the tibia contact surface. In some such embodiments, the adjustment mechanism includes a sliding connection between the ramp and the superior portion, wherein moving the superior portion along the sliding connection changes the distance between the tendon contact surface and the tibia contact surface. In some or all such embodiments, the adjustment mechanism also has a lock with a first configuration adapted to hold a position of the superior portion with respect to the inferior portion and a second configuration adapted to permit the superior portion to be moved with respect to the inferior portion.

In some embodiments, the adjustment mechanism comprises a column fixed to, and extending up from, the inferior portion, and a plurality of connection slots to which the superior portion can be selectively engaged. In some such embodiments, the adjustment mechanism also has a tab supported by the superior portion and biased by a spring, the spring biasing the tab into one of the connection slots to prevent relative movement between the superior portion and the inferior portion, the tab being movable against the spring out of the one of the connection slots to permit movement of the superior portion with respect to the inferior portion. The orthopedic implant may also have a button operatively connected to the tab to move the tab against the spring. In other such embodiments, the adjustment mechanism further has a rotatable connection between the superior portion and the column, the rotatable connection having a first position permitting movement of the superior portion to a second connection position and a second position preventing movement of the superior portion from the first connection position to the second connection position.

In some embodiments, the orthopedic implant has a sealed cavity disposed between the superior portion and the inferior portion, the adjustment mechanism comprising a port in fluid communication with the sealed cavity.

Another aspect of the invention provides a method for repositioning a patellar tendon of a patient. In some embodiments, the method includes the steps of inserting an orthopedic implant in a first configuration between the patellar tendon and a tibia; engaging a tibia contact surface of an inferior portion of the orthopedic implant with the tibia; engaging a tendon contact surface of a superior portion of the orthopedic implant with the patellar tendon; changing a distance between the tendon contact surface and the tibia contact surface; and changing a position of the patellar tendon

In some embodiments, the step of changing a distance between the tendon contact surface and the tibia contact surface is performed after the steps of engaging the tibia contact surface with the tibia and engaging the tendon contact surface with the patellar tendon. In some such embodiments, the changing step includes the step of inserting a tool through a skin opening to engage an adjustment mechanism. Such methods may also include the step of piercing the skin with the tool.

In some embodiments, the step of changing a distance between the tendon contact surface and the tibia contact surface includes the step of moving the superior portion along a ramp.

In some embodiments in which a first end of the superior portion is connected to the inferior portion with a hinge or pivot, the step of changing a distance between the tendon contact surface and the tibia contact surface includes the step of moving a second end of the superior portion about the hinge or pivot.

In some embodiments, the step of changing a distance between the tendon contact surface and the tibia contact surface comprises changing a height of a piston extending between the superior portion and the inferior portion, such as by changing the height of the piston comprises adding or removing fluid from an interior of the piston or rotating a ring engaged with threads on the piston.

In some embodiments, the step of changing a distance between the tendon contact surface and the tibia contact surface includes the step of changing a position of a cam disposed between the superior portion and the inferior portion.

In embodiments in which the orthopedic implant has a column disposed between the inferior portion and the superior portion, the step of changing a distance between the tendon contact surface and the tibia contact surface may include the step of disengaging a tab from a slot in the column. In some such embodiments, the step of disengaging a tab from a slot in the column includes the step of moving the tab against a spring, and the step of changing a distance between the tendon contact surface and the tibia contact surface includes the step of permitting the spring to move the tab back into a slot of the column. In other such embodiments, the step of disengaging a tab from a slot in the column includes the step of rotating the superior portion with respect to the inferior portion.

In embodiments in which the orthopedic implant has an expandable cavity disposed between the tendon contact surface and the tibia contact surface, the step of changing a distance between the tendon contact surface and the tibia contact surface may include the step of adding or removing fluid from the expandable cavity.

In some embodiments, the step of changing a distance between the tendon contact surface and the tibia contact surface is performed before the steps of engaging the tibia contact surface with the tibia and engaging the tendon contact surface with the patellar tendon.

Some embodiments include the further step of locking the orthopedic implant after changing the distance between the tendon contact surface and the tibia contact surface.

Additional examples are provided as well.

In Example 1, an orthopedic implant comprises a base section having a bone contact surface configured to be positionable in contact with a bone, a top section opposite to the base section and disposed between the patellar tendon and the base section, the top section having a tendon contact surface configured to change an elevation of a patellar tendon by lifting the patellar, a joint rotatably coupling the top section to the base section at a lateral side or a medial side of the implant, a fixation mechanism adapted to attach the orthopedic implant to the bone, and an adjustment mechanism adapted to change a distance between the tendon contact surface and the bone contact surface via rotation at the joint such that the tendon contact surface is configured to urge the patellar tendon medially or laterally.

Example 2 relates to the orthopedic implant according to Example 1, wherein the patellar tendon is urged medially or laterally when the adjustment mechanism causes the change in the distance between the tendon contact surface and the bone contact surface via rotation at the joint such that the patellar tendon is also caused to rotate around its longitudinal axis.

Example 3 relates to the orthopedic implant according to Example 2, wherein the rotation of the patellar tendon around its longitudinal axis causes rotation of the patella.

Example 4 relates to the orthopedic implant according to Example 1, wherein the joint rotatably couples the top section to the base section at the medial side, and wherein the adjustment mechanism is disposed near the lateral side.

Example 5 relates to the orthopedic implant according to Example 4, wherein actuation of the adjustment mechanism to urge the tendon contact surface away from the bone contact surface causes the patellar tendon to move medially.

Example 6 relates to the orthopedic implant according to Example 4, wherein actuation of the adjustment mechanism to urge the tendon contact surface toward the bone contact surface causes the patellar tendon to move laterally.

In Example 7, an orthopedic implant comprises a base section having a bone contact surface configured to be positionable in contact with a bone, a top section opposite to the base section having a tendon contact surface configured to change a position of a patellar tendon by lifting or tilting the patellar tendon when the bone contact surface is engaged with the bone, a fixation mechanism adapted to attach the orthopedic implant to the bone, and an adjustment mechanism adapted to change a distance between the tendon contact surface and the bone contact surface, wherein the adjustment mechanism comprises a piston disposed between the top section and the base section and near a lateral side or a medial side of the implant such that the tendon contact surface is configured to urge the patellar tendon medially or laterally.

Example 8 relates to the orthopedic implant according to Example 7, wherein the patellar tendon is urged medially or laterally when the adjustment mechanism causes the change in the distance between the tendon contact surface and the bone contact surface such that the patellar tendon is also caused to rotate around its longitudinal axis.

Example 9 relates to the orthopedic implant according to Example 8, wherein the rotation of the patellar tendon around its longitudinal axis causes rotation of the patella.

Example 10 relates to the orthopedic implant according to Example 7, wherein the piston is disposed near the lateral side of the implant.

Example 11 relates to the orthopedic implant according to Example 10, wherein actuation of the piston to urge the tendon contact surface away from the bone contact surface causes the patellar tendon to move medially.

Example 12 relates to the orthopedic implant according to Example 10, wherein actuation of the piston to urge the tendon contact surface toward the bone contact surface causes the patellar tendon to move laterally.

Example 13 relates to the orthopedic implant according to Example 7, wherein the adjustment mechanism further comprises a port fluidly connected with an interior of the piston through which fluid can be added or removed from the interior of the piston.

Example 14 relates to the orthopedic implant according to Example 7, wherein the piston comprises a threaded connection between a superior piston element and an inferior piston element.

In Example 15, an orthopedic implant comprises a base section having a bone contact surface configured to be positionable in contact with a bone, a top section opposite to the base section and having a tendon contact surface configured to change a position of a patellar tendon by elevating the patellar tendon when the bone contact surface is engaged with the bone, a fixation mechanism adapted to attach the orthopedic implant to the bone, and an adjustment mechanism adapted to change a distance between the tendon contact surface and the bone contact surface at a lateral side or a medial side of the implant such that the tendon contact surface is configured to urge the patellar tendon medially or laterally, wherein the adjustment mechanism comprises a rotatable cam disposed between the top section and the base section.

Example 16 relates to the orthopedic implant according to Example 15, wherein the patellar tendon is urged medially or laterally when the adjustment mechanism causes the change in the distance between the tendon contact surface and the bone contact surface such that the patellar tendon is also caused to rotate around its longitudinal axis.

Example 17 relates to the orthopedic implant according to Example 16, wherein the rotation of the patellar tendon around its longitudinal axis causes rotation of the patella.

Example 18 relates to the orthopedic implant according to Example 15, wherein the rotatable cam is disposed near the lateral side of the implant.

Example 19 relates to the orthopedic implant according to Example 18, wherein

actuation of the rotatable cam to urge the tendon contact surface away from the bone contact surface causes the patellar tendon to move medially.

Example 20 relates to the orthopedic implant according to Example 18, wherein actuation of the rotatable cam to urge the tendon contact surface toward the bone contact surface causes the patellar tendon to move laterally.

While multiple embodiments are disclosed, still other embodiments will become apparent to those skilled in the art from the following detailed description, which shows and describes various illustrative implementations. As will be realized, the various embodiments herein are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is an end view of an orthopedic implant according to an embodiment of the invention in an unextended position.

FIG. 1B is a side view of the orthopedic implant of FIG. 1A.

FIG. 2A is an end view of the orthopedic implant of FIG. 1 in an extended position.

FIG. 2B is a side view of the orthopedic implant of FIG. 2A

FIG. 3 shows the orthopedic implant of FIGS. 2A-2B in place on a patient.

FIG. 4 is a perspective view of an orthopedic implant according to another embodiment of the invention.

FIG. 5 is a perspective view of an orthopedic implant according to yet another embodiment of the invention.

FIG. 6 is a perspective view of an orthopedic implant according to still another embodiment of the invention.

FIG. 7 is a perspective view of the orthopedic implant of FIG. 6 and an adjustment tool.

FIG. 8 is a perspective view of an orthopedic implant according to another embodiment of the invention.

FIG. 9 is a perspective view part of the height adjustment mechanism of the orthopedic implant of FIG. 8.

FIG. 10 shows the orthopedic implant of FIG. 8 after implantation on the patient.

FIG. 11 is a perspective view of the orthopedic implant of FIG. 8 and an adjustment tool.

FIG. 12 is an exploded view of an orthopedic implant according to yet another embodiment of the invention.

FIG. 13 is a top view of the orthopedic implant of FIG. 12 in a locked position.

FIG. 14 is a top view of the orthopedic implant of FIG. 12 in an unlocked position.

FIG. 15 is a perspective view of an orthopedic implant according to still another embodiment of the invention.

FIG. 16 is a perspective view of the orthopedic implant of FIG. 15 in place on a patient.

FIG. 17 is a perspective view of an orthopedic implant according to another embodiment of the invention.

FIG. 18A is a side view of the orthopedic implant of FIG. 17 with the piston in a raised position, according to one embodiment.

FIG. 18B is a side view of the orthopedic implant of FIG. 17 with the piston in a lowered position, according to one embodiment.

FIG. 19 is a perspective view of an orthopedic implant according to yet another embodiment of the invention.

FIG. 20A is a side view of the orthopedic implant of FIG. 19 with the piston in a raised position, according to one embodiment.

FIG. 20B is a side view of the orthopedic implant of FIG. 19 with the piston in a lowered position, according to one embodiment.

FIG. 21 is a side view of an orthopedic implant according to a further embodiment of the invention.

FIG. 22 is a perspective view of the orthopedic implant of FIG. 21 along with a tool that can be used to adjust the implant, according to one embodiment.

FIG. 23A is a perspective view of an orthopedic implant according to yet another embodiment of the invention.

FIG. 23B is a side view of the orthopedic implant of FIG. 23B, according to one embodiment.

FIG. 24 is a perspective view of an orthopedic implant according to another alternative embodiment of the invention.

FIG. 25 is a side view of the orthopedic implant of FIG. 24 along with a tool that can be used to adjust the implant, according to one embodiment.

### DETAILED DESCRIPTION

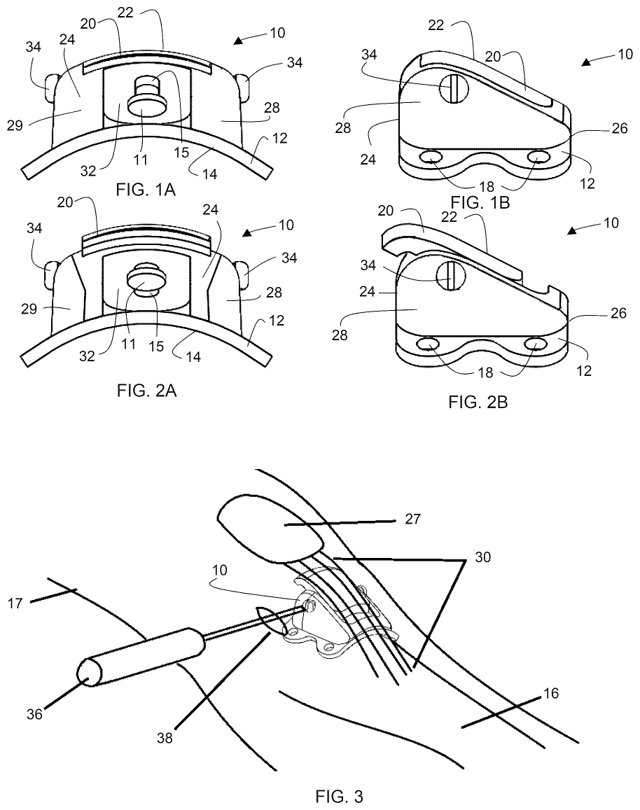

FIGS. 1-3 show one embodiment of an adjustable orthopedic implant for use in adjusting the height of a patellar tendon. Orthopedic implant 10 has an inferior portion 12 with a tibia contact surface 14 adapted to engage the patient's tibia 16 in the patient's leg 17. Holes 18 on both sides of the inferior portion are sized to receive screws or other fixation devices to attach the implant to the tibia 16.

The superior portion 20 of the implant has a tendon contact surface 22 on its top side. Two side portions 28 and 29 extend between the inferior portion and the superior portion. In this embodiment, the anterior end 24 of the implant 10 is taller than the posterior end 26 of the implant. When in place on the patient, the anterior end 24 is placed closer to the patient's patella 27 than the posterior end 26 is, as shown in FIG. 3. The patellar tendon 30 rests on the tendon contact surface 22.

Superior portion 20 may be extended or retracted along a ramp 32 extending between the side portions 28 and 29 to change the distance between the tibia contact surface and the tendon contact surface by changing the effective height of the anterior end of the implant in order to provide the desired upward lift of the patellar tendon. A pin 11 extending from superior portion 20 through a slot 15 in the ramp 32 limits the motion of superior portion 20 along the ramp 32. Thereafter, locks 34 (such as, e.g., a movable screw) may be engaged to prevent further movement. The implant's effective height may be set prior to implantation, or, as shown in FIG. 3, the position of the superior portion may be changed after implantation. A tool 36 may be inserted through an incision 38 to access the locks 34. The orthopedic implant of FIGS. 1-3 may be made of any suitable material (e.g., rigid metals and/or plastics).

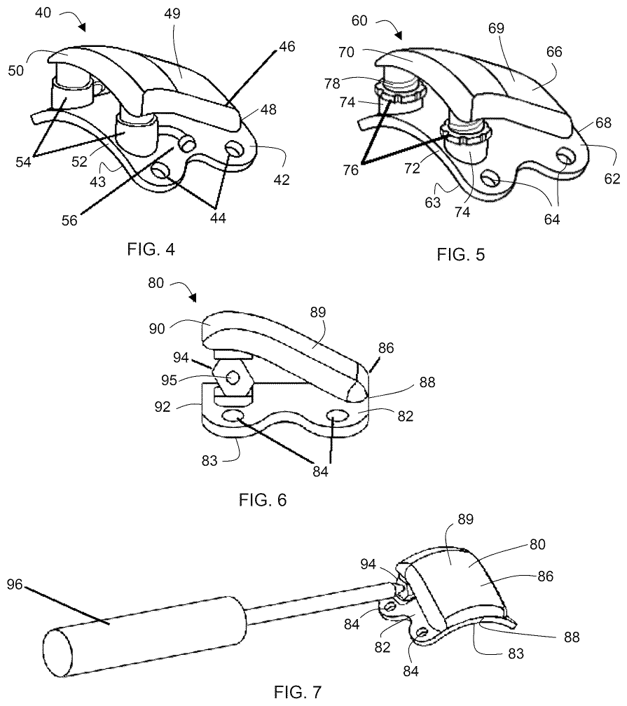

FIGS. 4-7 show embodiments of orthopedic implants according to this invention in which the superior portion connects to the inferior portion via a pivot or hinge. Changes in the implant height change the distance between the tendon contact surface and the tibia contact surface.

The orthopedic implant 40 of FIG. 4 has an inferior portion 42 with a tibia contact surface 43 adapted to engage a patient's tibia. Holes 44 on both sides of the inferior portion are sized to receive screws or other fixation devices to attach the implant to the tibia. Superior portion 46 of orthopedic implant 40 is connected to the inferior portion 42 via a pivot or hinge at the posterior end 48 of the implant. Superior portion 46 has a tendon contact surface 49 on its top side. The anterior end 50 of the superior portion 46 may be raised or lowered with respect to the anterior end 52 of the inferior portion 42 by increasing or decreasing the height of pistons 54. Fluid (such as saline solution) may be added to interior chambers of the pistons 54 through an access ports 56 (only one of which is shown in FIG. 4) to increase the height of the pistons, thereby rotating the anterior end of the superior portion about the pivot or hinge, increasing the height of the anterior end of the implant and raising the patellar tendon. Fluid may be extracted from the pistons' interior chambers to decrease their height, thereby decreasing the height of the anterior end of the implant and decreasing the amount of patellar tendon lift. This adjustment to the height of the implant may be performed prior to implantation of the implant and affixation of the implant to the tibia. The implant's height may also be adjusted after implantation and affixation by inserting the fluid line through an incision in the patient's leg and connecting the fluid line with the access ports 56. The orthopedic implant of FIG. 4 may be made of any suitable material (e.g., rigid metals and/or plastics).

FIG. 5 shows an orthopedic implant 60 with an inferior portion 62 and a tibia contact surface 63 adapted to engage a patient's tibia. Holes 64 on both sides of the inferior portion are sized to receive screws or other fixation devices to attach the implant to the tibia. Superior portion 66 of orthopedic implant 60 is connected to the inferior portion 62 via a pivot or hinge at the posterior end 68 of the implant. Superior portion 66 has a tendon contact surface 69 on its top side. The anterior end 70 of the superior portion 66 may be raised or lowered with respect to the anterior end 72 of the inferior portion 62 by increasing or decreasing the height of pistons 74. In this embodiment, the pistons' heights are adjusted by rotating a threaded ring 76 engaged with threads 78 in pistons 74. Once again, an increase in the height of the pistons rotates the anterior end of the superior portion about the pivot or hinge, increasing the height of the anterior end of the implant and raising the patellar tendon. A decrease in the pistons' height decreases the height of the anterior end of the implant and lowers the patellar tendon. This adjustment to the height of the implant, and consequent change in the distance between the tendon contact surface and the tibia contact surface, may be performed prior to or after implantation of the implant and affixation of the implant to the tibia. The orthopedic implant of FIG. 5 may be made of any suitable material (e.g., rigid metals and/or plastics).

The orthopedic implant 80 shown in FIGS. 6 and 7 has an inferior portion 82 and a tibia contact surface 83 adapted to engage a patient's tibia. Holes 84 on both sides of the inferior portion are sized to receive screws or other fixation devices to attach the implant to the tibia. Superior portion 86 of orthopedic implant 80 is connected to the inferior portion 82 via a pivot or hinge at the posterior end 88 of the implant. Superior portion 86 has a tendon contact surface 89 on its top side. The anterior end 90 of the superior portion 86 may be raised or lowered with respect to the anterior end 92 of the inferior portion 82 by rotating a cam 94 by engaging a hole 95 in cam 94 with a tool 96. Once again, a change in the implant's height changes the distance between the tendon contact surface and the tibia contact surface. An increase in the implant's height on the anterior end raises the patellar tendon, and a decrease in the height of the anterior end of the implant lowers the patellar tendon. This adjustment to the height of the implant may be performed prior to or after implantation of the implant and affixation of the implant to the tibia. The orthopedic implant of FIGS. 6-7 may be made of any suitable material (e.g., rigid metals and/or plastics).

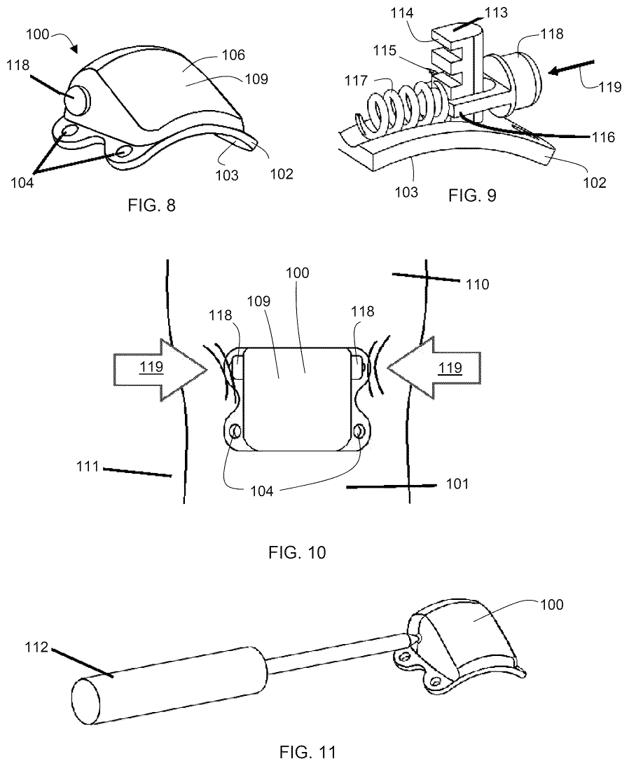

FIGS. 8-11 show yet another embodiment of an orthopedic implant according to this invention. Implant 100 has an inferior portion 102 and a tibia contact surface 103 adapted to engage a patient's tibia. Holes 104 on both sides of the inferior portion are sized to receive screws or other fixation devices to attach the implant to the tibia. Superior portion 106 has a tendon contact surface 109 on its top side. Two columns 113 (one of which is shown in FIG. 9) are affixed to the inferior portion 102 and extend upward within the superior portion 106. Columns 113 have alternating ridges 114 and slots 115. A tab 116 operatively connected to, and supported by, superior portion 106 is biased into one of the slots 115 by a spring 117. A button 118 connected to the tab 116 extends to the exterior of superior portion 106. When buttons 118 are pressed (as shown by arrows 119) to move tab 116 against the action of springs 117, tabs 116 move out of their respective slots 115, enabling the superior portion 106 to be raised or lowered with respect to the inferior portion 102. Releasing the buttons 118 permits the springs 117 to move their respective tabs 116 into slots 115 on columns 113 corresponding to the position to which the superior portion has been moved. The buttons may be depressed by a user's fingers or by using a tool 112, as shown in FIG. 11. As in the other embodiments, a change in the implant's height changes the distance between the tendon contact surface and the tibia contact surface. An increase in the implant's height raises the patellar tendon, and a decrease in the implant's height lowers the patellar tendon. This adjustment to the height of the implant may be performed prior to or after implantation of the implant and affixation of the implant to the tibia. For example, FIG. 10 shows implant 100 in place on the tibia 101 below the knee 110 in the patient's leg 111. The orthopedic implant of FIGS. 8-11 may be made of any suitable material (e.g., rigid metals and/or plastics).

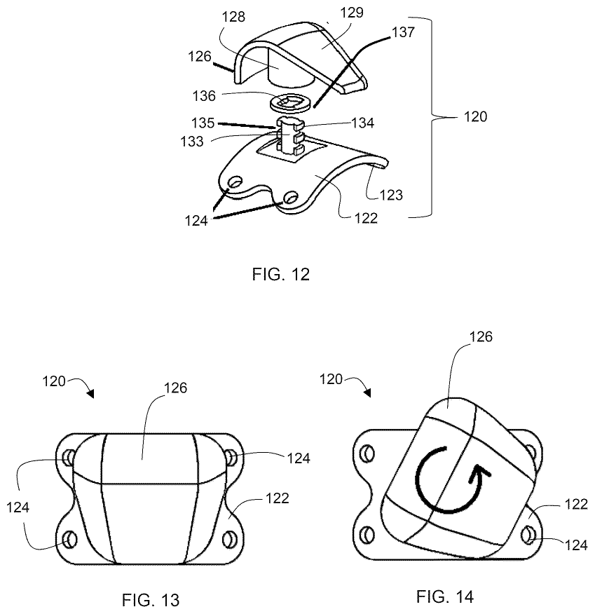

The orthopedic implant 120 shown in FIGS. 12-14 has an inferior portion 122 and a tibia contact surface 123 adapted to engage a patient's tibia. Holes 124 on both sides of the inferior portion are sized to receive screws or other fixation devices to attach the implant to the tibia. Superior portion 126 has a tendon contact surface 129 on its top side. A column 133 is affixed to the inferior portion 122 and extends upward within the superior portion 126. Column 133 has corresponding ridges 134 and slots 135 on two opposing sides and no ridges or tabs on the other two opposing sides. Extending down from an inner surface of superior portion 126 is a hollow structure 128 with an oval-shaped opening 136 facing the inferior portion 122. As shown in the exploded view of FIG. 12 (in which the superior portion 126 is partially cut away for visibility), opening 136 is formed in a separate ring 137 that attaches to the bottom end of hollow structure 128. Other embodiments omit the ring, and the oval-shaped opening is integral with structure 128. Superior portion 126 may be rotated with respect to inferior portion 122, as shown in FIG. 14, to line up the long axis of oval-shaped opening 136 with ridges 134, thereby enabling superior portion 126 to be raised or lowered with respect to inferior portion 122. When the desired implant height is achieved, superior portion 126 is rotated back to the position shown in FIG. 13, which aligns the short axis of oval-shaped opening with the ridges 134 so that portions of the ring 137 on either side of the short axis sit between adjacent ridges, thereby locking the superior portion 126 in place. The portion of column 133 above the slots to which ring is engaged extends into the cavity of structure 128. As in the other embodiments, a change in the implant's height changes the distance between the tendon contact surface and the tibia contact surface. An increase in the implant's height raises the patellar tendon, and a decrease in the implant's height lowers the patellar tendon. This adjustment to the height of the implant may be performed prior to or after implantation of the implant and affixation of the implant to the tibia (by, e.g., manipulating the implant through the skin). The orthopedic implant of FIGS. 12-14 may be made of any suitable material (e.g., rigid metals and/or plastics).

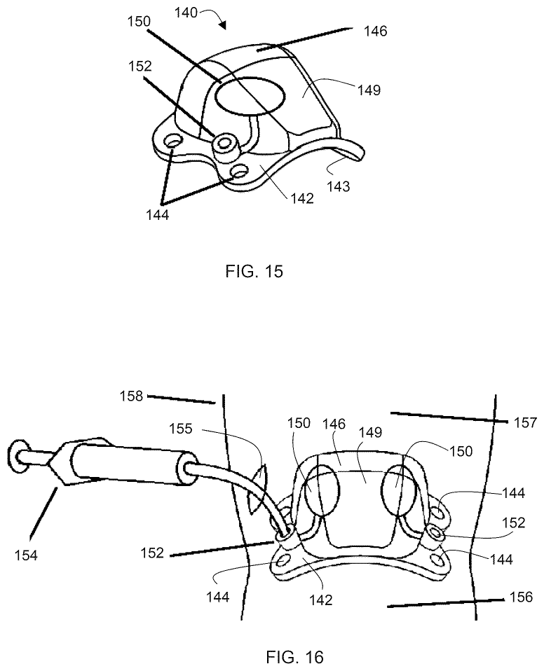

FIGS. 15 and 16 show yet another embodiment of an orthopedic implant according to this invention. Orthopedic implant 140 has an inferior portion 142 and a tibia contact surface 143 adapted to engage a patient's tibia. Holes 144 on both sides of the inferior portion are sized to receive screws or other fixation devices to attach the implant to the tibia. Superior portion 146 has a tendon contact surface 149 on its top side. In this embodiment, all or part of the superior portion 146 is made of a flexible material. One or more cavities 150 within the superior portion 146 beneath the tendon contact surface 149 communicate with sealing access ports 152. Fluid (e.g., air or saline) may be added or withdrawn from cavities 150 with a tool 154 engaged with access ports 152 to change the height of superior portion 146 with respect to the inferior portion 142, thereby changing the distance between the tendon contact surface 149 and tibia contact surface 143. This adjustment to the height of the implant may be performed prior to or after implantation of the implant and affixation of the implant to the tibia. As shown in FIG. 16, the tool 154 may be inserted through an incision 155 in the patient's leg 158 to the implant 140 in place on the patient's tibia 156 below the knee 157. The use of two cavities, as shown in FIG. 16, enables the height of the implant to be adjusted differently on the medial and lateral sides.

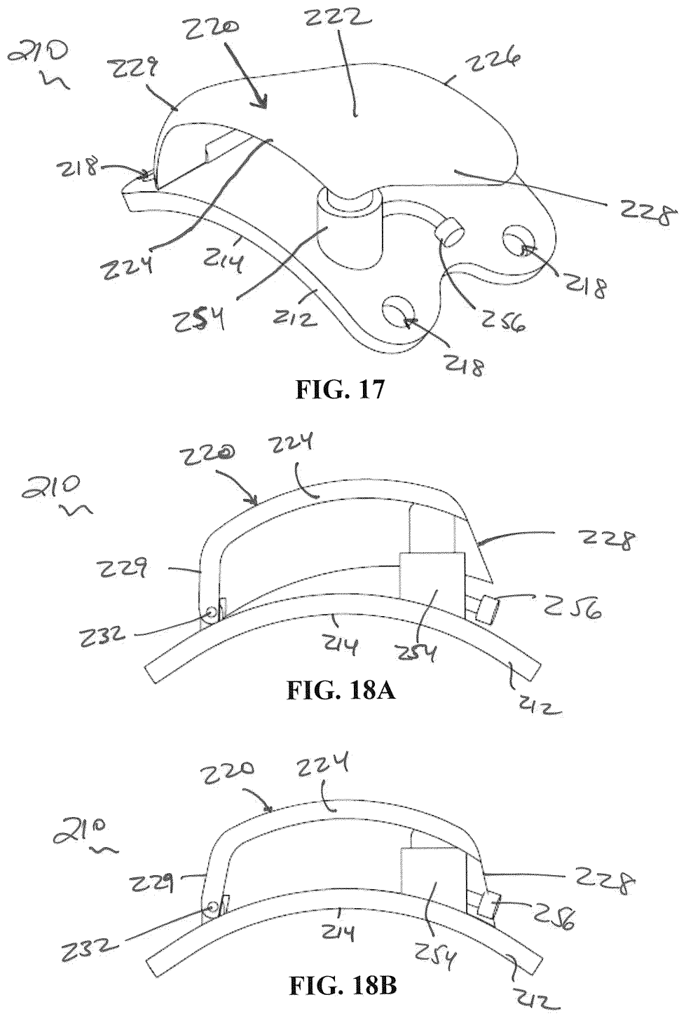

According to other implementations, certain adjustable orthopedic implant versions are provided herein that allow for adjusting the height of the superior portion of the implant at one side (rather than by adjusting the height of the anterior end). In this fashion, instead of adjusting the height of the patellar tendon, or instead of adjusting solely the height or elevation of the tendon, the various implant embodiments described below provide for adjustment of the position of the patellar tendon in the lateral or medial direction (to one side or the other, which can also be referred to herein as “realignment”). Hence, the adjustment of the height of the superior portion at one side or the other can cause not only the adjustment of the height or elevation of the patellar tendon, but also the realignment of the tendon medially or laterally, and in some instances, can also cause the rotation of the patellar tendon along its longitudinal axis in either of those directions as well. In addition, according to some implementations, such rotation of the tendon can also cause the rotation of the patella. Thus, each of the various embodiments discussed below (with respect to FIGS. 17-25) that allow for adjustment of the height of the superior portion at one side or the other can cause such movement of the patellar tendon and the patella.

For example, FIGS. 17-18B depict one implementation of an adjustable implant 210 for use in adjusting the position of the patellar tendon in the lateral or medial direction. The implant 210 has an inferior portion 212 with a tibia contact surface 214 adapted to engage the patient's tibia (such as tibia 16 as discussed below) in the patient's leg (such as leg 17). Holes 218 on both sides of the inferior portion 212 are sized to receive screws or other fixation devices to attach the implant 210 to the tibia. The superior portion 220 of the implant 210 has a tendon contact surface 222 on its top side. Two side portions 228 and 229 extend between the inferior portion 212 and the superior portion 220. In this embodiment, the anterior end 224 of the implant 210 is taller than the posterior end 226 of the implant. When in place on the patient, the anterior end 224 is placed closer to the patient's patella (such as patella 27 discussed above) than the posterior end 226 is (as shown in FIG. 3 above relating to a different embodiment). The patellar tendon (such as tendon 30 above) rests on the tendon contact surface 222.

The contact surface 222 at the first side 228 can be raised or lowered around a joint 232 to change the distance between the contact surface 222 at the first side 228 and the tibia contact surface 214, thereby changing the effective height of the contact surface 222 at the first side 228 in order to urge the patellar tendon to move and/or rotate between the first and second sides 228, 229. The joint 232 in this embodiment is positioned at the coupling of the second side 229 and the inferior portion 212 such that the entire superior portion 220, including the first side 228, the contact surface 222, and the second side 229, can rotate around the joint 232 in relation to the inferior portion 212.

In this exemplary implementation, the implant 210 has an actuable piston 254 operably coupled to the inferior portion 212 and the superior portion 220 as shown. As such, piston 254 can be actuated such that the contact surface 222 at the first side 228 can be raised or lowered in relation to the inferior portion 212 by increasing or decreasing the height of the piston 254. In one specific version, fluid (such as saline solution or any other known fluid) may be added to an interior chamber of the piston 254 through an access port 256 to increase the height of the piston 254, thereby rotating the first side 228 of the superior portion 220 about the joint 232 and thus increasing the height of the first side 228. By raising the height of the first side 228, the implant 210 can urge the tendon disposed in contact with the contact surface 222 toward the second side 229. Further, fluid can be extracted from the interior chamber of the piston 254 to decrease its height, thereby decreasing the height of the first side 228. By lowering the height of the first side 228, the implant 210 can urge the tendon disposed in contact with the contact surface 222 toward the first side 228. This adjustment to the height of the first side 222 of the superior portion 220 can be performed prior to implantation of the implant 210 and affixation of the implant 210 to the tibia. Alternatively, the height can also be adjusted after implantation and affixation by inserting a fluid line through an incision in the patient's leg and connecting the fluid line with the access port 256. As such, the height of the implant 210 can be adjusted immediately after implantation, or it can be adjusted at a later time by connection to the access port 256.

One of ordinary skill understands that the orthopedic implant 210 of FIGS. 17-18B can be made of any suitable material (including, for example, rigid metals and/or plastics). Further, the actuable piston 254 can be any known piston of any kind that can be incorporated into an orthopedic implant, including a hydraulic piston, for example. In addition, the joint 232 can be any known hinge, pivot, or any other component or mechanism that allows for rotation of the superior portion 220 in relation to the inferior portion 212 in a fashion similar to that described above.

In certain alternative embodiments, the implant 210 can have a contact surface 222 that is raised and lowered at the second side 229 instead of the first side 228. That is, the joint 232 is disposed between the first side 228 and the inferior portion 212 such that the first side 228 is rotatably coupled to the inferior portion 212 via the joint 232. Further, in such embodiments, the piston 254 is disposed between the second side 229 and the inferior portion 212 such that the piston 254 can raise or lower the second side 229 in relation to the inferior portion 212.

It is noted that other terms can be used interchangeably with the terms set forth above for the various aspects of the exemplary embodiments as set forth herein. Thus, while the sides 228, 229 as discussed above are referred to as first and second sides 228, 229, they can also be referred to as the lateral side 228 and the medial side 229. That is, the sides of the implant 210 (or any other implant implementation as set forth herein) can identified based on the intended positioning of the implant 210 when attached to the patient's right tibia, with the lateral side 228 facing toward the lateral side of the patient's right leg (away from the left leg), and the medial side 229 facing toward the medial side of the patient's right leg (toward the left leg). Further, while the other two sides of the implant 210 are referred to as the anterior end 224 and the posterior end 226 above, they can also be referred to as the proximal side 224 and the distal side 226. In addition, the superior portion 220 can be referred to as the upper or top section 220 and the inferior portion 212 can be referred to as the bottom or base section 212.

However, the names of the sides are provided solely for convenience and ease of understanding and are not limiting. In alternative descriptions, the proximal side 224 can be a first side 224, the distal side 226 can be a second side 226, the lateral side 228 can be a third side 228, and the medial side 229 can be a fourth side 229.

The various interchangeable terms as discussed above can also be used with reference to any of the various implant embodiments disclosed or contemplated herein.

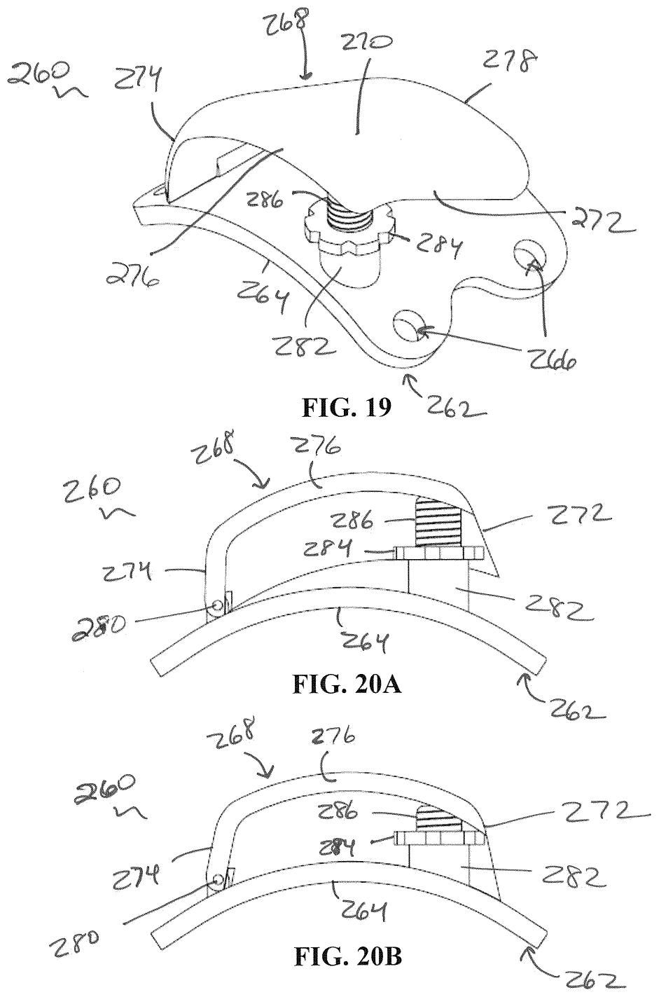

According to another embodiment, FIGS. 19-20B depict yet another implementation of an adjustable implant 260 for use in adjusting the position of the patellar tendon in the lateral or medial direction. The implant 260 has an inferior portion 262 with a tibia contact surface 264 adapted to engage the patient's tibia (such as tibia 16 as discussed below) in the patient's leg (such as leg 17). Holes 266 on both sides of the inferior portion 262 are sized to receive screws or other fixation devices to attach the implant 260 to the tibia. The superior portion 268 of the implant 260 has a tendon contact surface 270 on its top side. Two side portions 272 and 274 extend between the inferior portion 262 and the superior portion 268. In this embodiment, the anterior end 276 of the implant 260 is taller than the posterior end 278 of the implant. When in place on the patient, the anterior end 276 is placed closer to the patient's patella (such as patella 27 discussed above) than the posterior end 278 is (as shown in FIG. 3 above relating to a different embodiment). The patellar tendon (such as tendon 30 above) rests on the tendon contact surface 270.

In a similar fashion to the implant 210 discussed above, the contact surface 270 at the first side 272 can be raised or lowered around a joint 280 to change the distance between the contact surface 270 at the first side 272 and the tibia contact surface 264, thereby changing the effective height of the contact surface 270 at the first side 272 in order to urge the patellar tendon to move and/or rotate between the first and second sides 272, 274. The joint 280 in this embodiment is positioned at the coupling of the second side 274 and the inferior portion 262 such that the entire superior portion 268, including the first side 272, the contact surface 270, and the second side 274, can rotate around the joint 280 in relation to the inferior portion 262.

In this exemplary implementation, the implant 260 has an actuable piston 282 operably coupled to the inferior portion 262 and the superior portion 268 as shown. As such, piston 282 can be actuated such that the contact surface 270 at the first side 272 can be raised or lowered in relation to the inferior portion 262 by increasing or decreasing the height of the piston 282. In one specific version, the actuable piston 282 is a mechanically actuable piston 282 having a threaded ring 284 engaged with threads 286 such that rotation of the ring 284 causes the piston 282 to increase or decrease in height. Thus, rotation of the ring 284 in one direction causes the height of the piston 282 to increase, thereby rotating the first side 272 of the superior portion 268 about the joint 280 and thus increasing the height of the first side 272. By raising the height of the first side 272, the implant 260 can urge the tendon disposed in contact with the contact surface 270 toward the second side 274. Further, the ring 284 can be rotated in the opposite direction to decrease its height, thereby decreasing the height of the first side 272. By lowering the height of the first side 272, the implant 260 can urge the tendon disposed in contact with the contact surface 270 toward the first side 272. This adjustment to the height of the first side 270 of the superior portion 268 can be performed prior to implantation of the implant 260 and affixation of the implant 260 to the tibia. Alternatively, the height can also be adjusted after implantation and affixation by inserting a tool through an incision in the patient's leg, coupling the tool with the ring 284, and rotating the ring 284 as desired. As such, the height of the implant 260 can be adjusted immediately after implantation, or it can be adjusted at a later time by connection to the ring 284.

In a further alternative, the height of the implant 260 can be adjusted without the need for an incision through the use of an external tool that relies on magnetic force or other non-contact force to cause rotation of the ring 284 within the patient.

One of ordinary skill understands that the orthopedic implant 260 of FIGS. 19-20B can be made of any suitable material (including, for example, rigid metals and/or plastics). Further, the actuable piston 282 can be any known mechanically-actuated piston of any kind that can be incorporated into an orthopedic implant. In addition, the joint 280 can be any known hinge, pivot, or any other component or mechanism that allows for rotation of the superior portion 268 in relation to the inferior portion 262 in a fashion similar to that described above.

In certain alternative embodiments, the implant 260 can have a contact surface 270 that is raised and lowered at the second side 274 instead of the first side 272. That is, the joint 280 is disposed between the first side 272 and the inferior portion 262 such that the first side 272 is rotatably coupled to the inferior portion 262 via the joint 280. Further, in such embodiments, the piston 282 is disposed between the second side 274 and the inferior portion 262 such that the piston 282 can raise or lower the second side 274 in relation to the inferior portion 262.

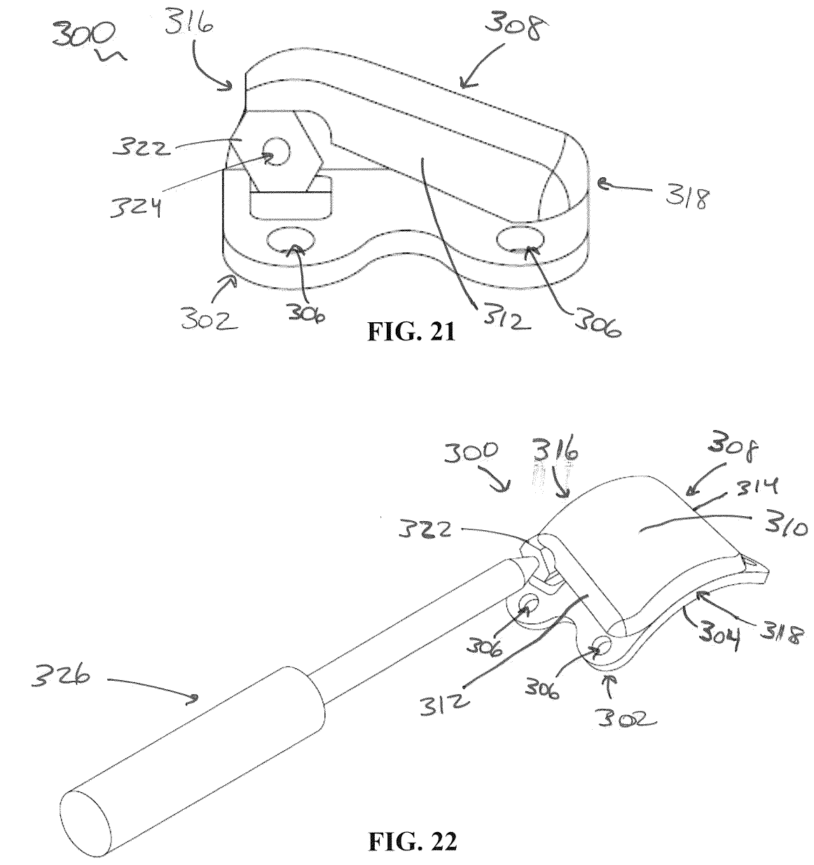

According to another embodiment, FIGS. 21-22 depict yet another implementation of an adjustable implant 300 for use in adjusting the position of the patellar tendon in the lateral or medial direction. The implant 300 has an inferior portion 302 with a tibia contact surface 304 adapted to engage the patient's tibia (such as tibia 16 as discussed below) in the patient's leg (such as leg 17). Holes 306 on both sides of the inferior portion 302 are sized to receive screws or other fixation devices to attach the implant 300 to the tibia. The superior portion 308 of the implant 300 has a tendon contact surface 310 on its top side. Two side portions 312 and 314 extend between the inferior portion 302 and the superior portion 308. In this embodiment, the anterior end 316 of the implant 300 is taller than the posterior end 318 of the implant. When in place on the patient, the anterior end 316 is placed closer to the patient's patella (such as patella 27 discussed above) than the posterior end 318 is (as shown in FIG. 3 above relating to a different embodiment). The patellar tendon (such as tendon 30 above) rests on the tendon contact surface 310.

In a similar fashion to the implants 210, 260 discussed above, the contact surface 310 at the first side 312 can be raised or lowered around a joint (not shown) to change the distance between the contact surface 310 at the first side 312 and the tibia contact surface 304, thereby changing the effective height of the contact surface 310 at the first side 312 in order to urge the patellar tendon to move and/or rotate between the first and second sides 312, 314. The joint (not shown) in this embodiment can be substantially similar to the joints 232, 280 discussed above, and thus can be positioned at the coupling of the second side 314 and the inferior portion 302 such that the entire superior portion 308, including the first side 312, the contact surface 310, and the second side 314, can rotate around the joint (not shown) in relation to the inferior portion 302.

In this exemplary implementation, the implant 300 has an actuable cam 322 operably coupled to the inferior portion 302 and the superior portion 308 as shown. As such, the cam 322 can be actuated such that the contact surface 310 at the first side 312 can be raised or lowered in relation to the inferior portion 302 by increasing or decreasing the height of the cam 322. In one specific version, the actuable cam 322 is a mechanically actuable cam or similar structure 322 having an opening 324 defined therethrough such that a tool 326 can be used to engage with the opening 324 and thereby rotate the cam 322, thereby causing the cam 322 to increase or decrease in height. Thus, rotation of the cam 322 in one direction causes the height of the cam 322 to increase, thereby rotating the first side 312 of the superior portion 308 about the joint 280 and thus increasing the height of the first side 312. By raising the height of the first side 312, the implant 300 can urge the tendon disposed in contact with the contact surface 310 toward the second side 314. Further, the cam 322 can be rotated in the opposite direction to decrease its height, thereby decreasing the height of the first side 312. By lowering the height of the first side 312, the implant 300 can urge the tendon disposed in contact with the contact surface 310 toward the first side 312. This adjustment to the height of the first side 310 of the superior portion 308 can be performed prior to implantation of the implant 300 and affixation of the implant 300 to the tibia. Alternatively, the height can also be adjusted after implantation and affixation by inserting the tool 326 through an incision in the patient's leg, coupling the tool 326 with the opening 324, and rotating the cam 322 as desired. As such, the height of the implant 300 can be adjusted immediately after implantation, or it can be adjusted at a later time by connection to the cam 322.

In a further alternative, the height of the implant 300 can be adjusted without the need for an incision through the use of an external tool that relies on magnetic force or other non-contact force to cause actuation of the cam 322 within the patient.

One of ordinary skill understands that the orthopedic implant 300 of FIGS. 21-22 can be made of any suitable material (including, for example, rigid metals and/or plastics). Further, the actuable cam 322 can be any known mechanically-actuated cam or similar structure of any kind that can be incorporated into an orthopedic implant. In addition, the joint (not shown) can be any known hinge, pivot, or any other component or mechanism that allows for rotation of the superior portion 308 in relation to the inferior portion 302 in a fashion similar to that described above.

In certain alternative embodiments, the implant 300 can have a contact surface 310 that is raised and lowered at the second side 314 instead of the first side 312. That is, the joint (not shown) is disposed between the first side 312 and the inferior portion 302 such that the first side 312 is rotatably coupled to the inferior portion 302 via the joint (not shown). Further, in such embodiments, the cam 322 is disposed between the second side 314 and the inferior portion 302 such that the cam 322 can raise or lower the second side 314 in relation to the inferior portion 302.

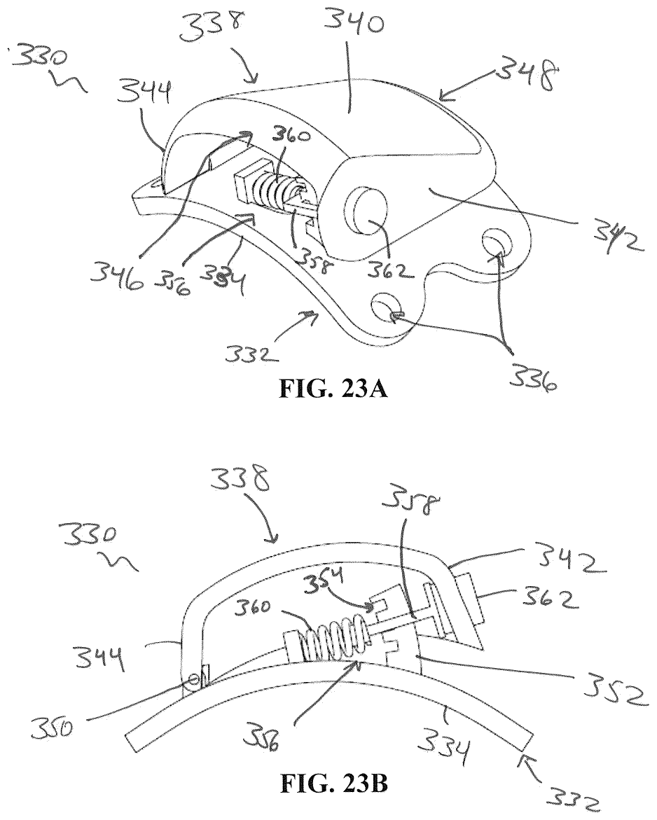

In accordance with a further implementation, FIGS. 23A-23B depict another adjustable implant 330 for use in adjusting the position of the patellar tendon in the lateral or medial direction. The implant 330 has an inferior portion 332 with a tibia contact surface 334 adapted to engage the patient's tibia (such as tibia 16 as discussed below) in the patient's leg (such as leg 17). Holes 336 on both sides of the inferior portion 332 are sized to receive screws or other fixation devices to attach the implant 330 to the tibia. The superior portion 338 of the implant 330 has a tendon contact surface 340 on its top side. Two side portions 342 and 344 extend between the inferior portion 332 and the superior portion 338. In this embodiment, the anterior end 346 of the implant 330 is taller than the posterior end 348 of the implant. When in place on the patient, the anterior end 346 is placed closer to the patient's patella (such as patella 27 discussed above) than the posterior end 348 is (as shown in FIG. 3 above relating to a different embodiment). The patellar tendon (such as tendon 30 above) rests on the tendon contact surface 340.

In a similar fashion to the implants 210, 260, 300 discussed above, the contact surface 340 at the first side 342 can be raised or lowered around a joint 350 to change the distance between the contact surface 340 at the first side 342 and the tibia contact surface 334, thereby changing the effective height of the contact surface 340 at the first side 342 in order to urge the patellar tendon to move and/or rotate between the first and second sides 342, 344. The joint 350 in this embodiment can be substantially similar to the joints 232, 280 discussed above, and thus is positioned at the coupling of the second side 344 and the inferior portion 332 as shown such that the entire superior portion 338, including the first side 342, the contact surface 340, and the second side 344, can rotate around the joint 350 in relation to the inferior portion 332.

In this exemplary implementation, the implant 330 has a column 352 attached to the inferior portion 332 and disposed between the inferior portion 332 and the superior portion 338 such that the column 352 extends toward the superior portion 338 as shown. Further, the column 352 has slots 354 defined in the side of the column 352 such that each slot 354 can receive the tab 358 of the tensioned adjustment mechanism 356. That is, the tensioned adjustment mechanism 356 is operably coupled to both the inferior portion 332 and the superior portion 338 such that movement of the adjustment mechanism 356 can cause movement of the superior portion 338 in relation to the inferior portion 332. Further, the tensioned adjustment mechanism 356 has a spring 360 or other tensioning mechanism that urges the tab 358 toward and into one of the slots 354, along with a button 362 moveably disposed through an opening in the first side 342 of the superior portion 338. The button 362 can be depressed by a user to overcome the spring 360 and urge the tab 358 away from the column 352 and thus out of the slot 354 in which it is disposed.

When the button 362 is pressed (urged toward the spring 360) to overcome the force of the spring 360 and move the tab 358, the tab 358 is urged out of the slot 354 in which it is positioned, enabling the superior portion 338 to be raised or lowered with respect to the inferior portion 332. Releasing the button 362 permits the spring 360 to urge the tab 358 back toward and into a selected slot 354 on the column 352 corresponding to the position to which the superior portion 338 has been moved. In various embodiments, the button 362 can be depressed by a user's fingers or by using a tool (such as a tool similar to the tool 326 discussed above, for example). Alternatively, any known tensioned adjustment mechanism can be used with the implant 330.

As such, the tensioned adjustment mechanism 356 can be actuated by depressing the button 362 such that the contact surface 340 at the first side 342 can be raised or lowered in relation to the inferior portion 332 by moving the tab 358 up or down in relation to the column 352. Thus, moving the tab 358 up (away from the inferior portion 332) in relation to the column 352 causes the first side 342 of the superior portion 338 to rotate about the joint 350 and thus increase the height of the first side 342. By raising the height of the first side 342, the implant 330 can urge the tendon disposed in contact with the contact surface 340 toward the second side 344. Further, the tab 358 can be moved downward (toward the inferior portion 332) to decrease the height of the first side 342. By lowering the height of the first side 342, the implant 330 can urge the tendon disposed in contact with the contact surface 340 toward the first side 342. This adjustment to the height of the first side 340 of the superior portion 338 can be performed prior to implantation of the implant 330 and affixation of the implant 330 to the tibia. Alternatively, the height can also be adjusted after implantation and affixation by inserting a tool through an incision in the patient's leg, depressing the button 362 with the tool, and adjusting the position of the tab 358 as desired. As such, the height of the implant 330 can be adjusted immediately after implantation, or it can be adjusted at a later time by depressing the button 362.

In a further alternative, the height of the implant 330 can be adjusted without the need for an incision through the use of an external tool that relies on magnetic force or other non-contact force to cause actuation of the mechanism 356 within the patient.

One of ordinary skill understands that the orthopedic implant 330 of FIGS. 23A-23B can be made of any suitable material (including, for example, rigid metals and/or plastics). Further, the adjustment mechanism 356 can be any known mechanically-actuated mechanism or similar structure of any kind that can be incorporated into an orthopedic implant. In addition, the joint 350 can be any known hinge, pivot, or any other component or mechanism that allows for rotation of the superior portion 338 in relation to the inferior portion 332 in a fashion similar to that described above.

In certain alternative embodiments, the implant 330 can have a contact surface 340 that is raised and lowered at the second side 344 instead of the first side 342. That is, the joint 350 is disposed between the first side 342 and the inferior portion 332 such that the first side 342 is rotatably coupled to the inferior portion 332 via the joint 350. Further, in such embodiments, the tensioned adjustment mechanism 356 is disposed such that the button 362 is disposed through an opening in the second side 344 such that the mechanism 356 can raise or lower the second side 344 in relation to the inferior portion 332.

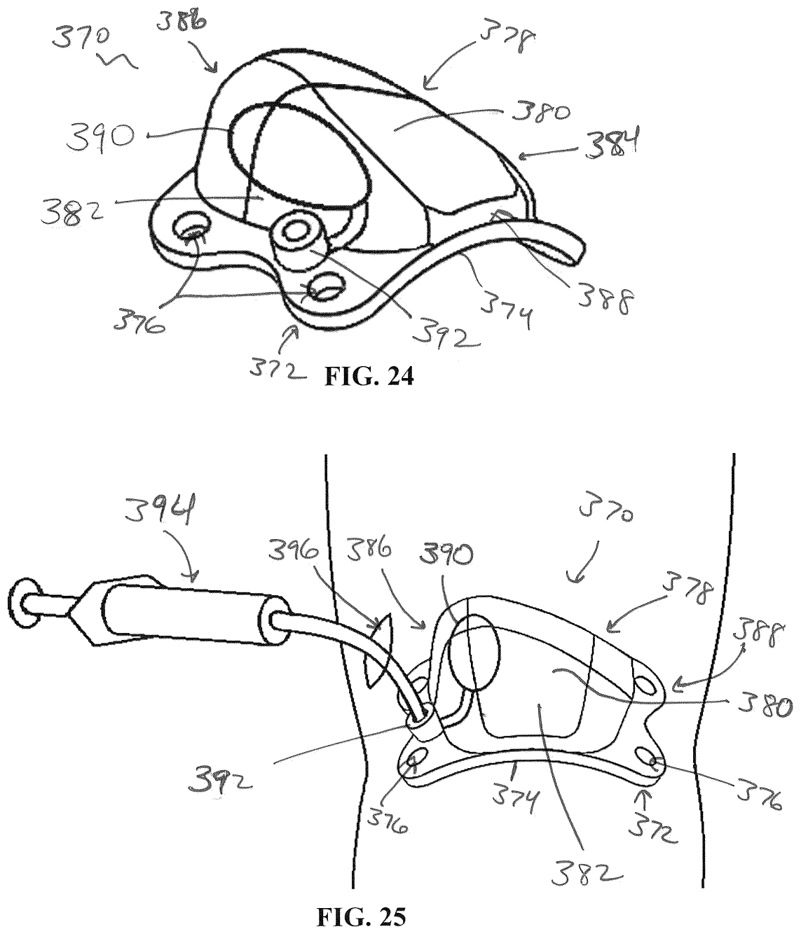

Alternatively, FIGS. 24-25 depict yet another embodiment of an adjustable implant 370 for use in adjusting the position of the patellar tendon in the lateral or medial direction. The implant 370 has an inferior portion 372 with a tibia contact surface 374 adapted to engage the patient's tibia (such as tibia 16 as discussed below) in the patient's leg (such as leg 17). Holes 376 on both sides of the inferior portion 372 are sized to receive screws or other fixation devices to attach the implant 370 to the tibia. The superior portion 378 of the implant 370 has a tendon contact surface 380 on its top side. Two side portions 382 and 384 extend between the inferior portion 372 and the superior portion 378. In this embodiment, the anterior end 386 of the implant 370 is taller than the posterior end 388 of the implant. When in place on the patient, the anterior end 386 is placed closer to the patient's patella (such as patella 27 discussed above) than the posterior end 388 is (as shown in FIG. 3 above relating to a different embodiment). The patellar tendon (such as tendon 30 above) rests on the tendon contact surface 380.

In a similar fashion to the implants 210, 260, 300, 330 discussed above, the contact surface 380 at the first side 382 can be raised or lowered to change the distance between the contact surface 380 at the first side 382 and the tibia contact surface 374, thereby changing the effective height of the contact surface 380 at the first side 382 in order to urge the patellar tendon to move and/or rotate between the first and second sides 382, 384. In this particular implementation, all or part of the superior portion 378 is made of a flexible material. Further, the implant 370 has an expandable cavity 390 disposed in the superior portion 378 beneath the tendon contact surface 380 that is in fluidic communication with a sealing access port 392. Fluid (e.g., air or saline or the like) may be added or withdrawn from the cavity 390 with a tool 394 that can be engaged with the access port 392 to change the height of the first side 382 with respect to the second side 384. Thus, inflation of the cavity 390 increases the height of the first side 382. By raising the height of the first side 382, the implant 370 can urge the tendon disposed in contact with the contact surface 380 toward the second side 384. Further, the cavity 390 can be deflated by removing fluid therefrom to decrease its height, thereby decreasing the height of the first side 382. By lowering the height of the first side 382, the implant 370 can urge the tendon disposed in contact with the contact surface 380 toward the first side 382. This adjustment to the height of the first side 380 of the superior portion 378 can be performed prior to implantation of the implant 370 and affixation of the implant 370 to the tibia. Alternatively, the height can also be adjusted after implantation and affixation by inserting the tool 394 through an incision 396 in the patient's leg, coupling the tool with the port 392, and inflating/deflating the cavity 390 as desired. As such, the height of the implant 370 can be adjusted immediately after implantation, or it can be adjusted at a later time by connection to the port 392.

One of ordinary skill understands that the orthopedic implant 370 of FIGS. 24-25 can be made of any suitable material (including, for example, rigid metals and/or plastics for most components and a flexible material for the superior portion 378 as discussed above). Further, the inflatable cavity 390 or balloon can be any known inflatable/deflatable component of any kind that can be incorporated into an orthopedic implant.

In certain alternative embodiments, the implant 370 can have a contact surface 380 that is raised and lowered at the second side 384 instead of the first side 382. That is, the cavity 390 is disposed in the superior portion 378 near the first side 382 such that the cavity 390 can raise or lower the second side 384 in relation to the inferior portion 372.

While the various implant and method embodiments herein are discussed in the context of the device being implanted between the tibia and patellar tendon, it should be noted that any and all of the implant and method embodiments herein can also be used to treat other joints in the human body. For example, the various implementations herein can be used to treat issues with the shoulder (such as implanting the device between the superior glenoid and the rotator cuff or between the proximal humerus and the rotator cuff, for example), the ankle (such as implanting the device between the Achilles tendon and the posterior calcaneus, for example), the spine (such as implanting the device between facet joints or in the disk space, for example), the hip (such as implanting the device between the pelvis and the gluteal muscles or between the greater trochanter and the gluteal tendons, for example), or other aspects of the knee (such as implanting the device between the iliotibial band and the lateral distal femur, for example). In other words, the various implant and method embodiments disclosed or contemplated herein are not limited to implantation between the patellar tendon and the tibia and can be used to unload other tendons and/or take pressure off other joints.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the words “about,” “approximately,” or “substantially,” even if the term does not expressly appear. The phrases “about,” “approximately,” or “substantially” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about, approximately, or substantially that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An orthopedic implant comprising:
(a) a base section having a bone contact surface configured to be positionable in contact with a bone;
(b) a top section opposite to the base section and disposed between the patellar tendon and the base section, the top section having a tendon contact surface configured to change an elevation of a patellar tendon by lifting the patellar tendon when the bone contact surface is engaged with the bone;
(c) a joint rotatably coupling the top section to the base section at a lateral side or a medial side of the implant;
(d) a fixation mechanism adapted to attach the orthopedic implant to the bone; and
(e) an adjustment mechanism adapted to change a distance between the tendon contact surface and the bone contact surface via rotation at the joint such that the tendon contact surface is configured to urge the patellar tendon medially or laterally.
(a) a base section having a bone contact surface configured to be positionable in contact with a bone;
(b) a top section opposite to the base section and disposed between the patellar tendon and the base section, the top section having a tendon contact surface configured to change an elevation of a patellar tendon by lifting the patellar tendon when the bone contact surface is engaged with the bone;
(c) a joint rotatably coupling the top section to the base section at a lateral side or a medial side of the implant;
(d) a fixation mechanism adapted to attach the orthopedic implant to the bone; and
(e) an adjustment mechanism adapted to change a distance between the tendon contact surface and the bone contact surface via rotation at the joint such that the tendon contact surface is configured to urge the patellar tendon medially or laterally.

2. The orthopedic implant of claim 1, wherein the patellar tendon is urged medially or laterally when the adjustment mechanism causes the change in the distance between the tendon contact surface and the bone contact surface via rotation at the joint such that the patellar tendon is also caused to rotate around its longitudinal axis.

3. The orthopedic implant of claim 2, wherein the rotation of the patellar tendon around its longitudinal axis causes rotation of the patella.

4. The orthopedic implant of claim 1, wherein the joint rotatably couples the top section to the base section at the medial side, and wherein the adjustment mechanism is disposed near the lateral side.

5. The orthopedic implant of claim 4, wherein actuation of the adjustment mechanism to urge the tendon contact surface away from the bone contact surface causes the patellar tendon to move medially.

6. The orthopedic implant of claim 4, wherein actuation of the adjustment mechanism to urge the tendon contact surface toward the bone contact surface causes the patellar tendon to move laterally.

7. An orthopedic implant comprising:
(a) a base section having a bone contact surface configured to be positionable in contact with a bone;
(b) a top section opposite to the base section having a tendon contact surface configured to change a position of a patellar tendon by lifting or tilting the patellar tendon when the bone contact surface is engaged with the bone;
(c) a fixation mechanism adapted to attach the orthopedic implant to the bone; and
(d) an adjustment mechanism adapted to change a distance between the tendon contact surface and the bone contact surface, wherein the adjustment mechanism comprises a piston disposed between the top section and the base section and near a lateral side or a medial side of the implant such that the tendon contact surface is configured to urge the patellar tendon medially or laterally.

8. The orthopedic implant of claim 7, wherein the patellar tendon is urged medially or laterally when the adjustment mechanism causes the change in the distance between the tendon contact surface and the bone contact surface such that the patellar tendon is also caused to rotate around its longitudinal axis.

9. The orthopedic implant of claim 8, wherein the rotation of the patellar tendon around its longitudinal axis causes rotation of the patella.

10. The orthopedic implant of claim 7, wherein the piston is disposed near the lateral side of the implant.

11. The orthopedic implant of claim 10, wherein actuation of the piston to urge the tendon contact surface away from the bone contact surface causes the patellar tendon to move medially.

12. The orthopedic implant of claim 10, wherein actuation of the piston to urge the tendon contact surface toward the bone contact surface causes the patellar tendon to move laterally.

13. The orthopedic implant of claim 7, wherein the adjustment mechanism further comprises a port fluidly connected with an interior of the piston through which fluid can be added or removed from the interior of the piston.

14. The orthopedic implant of claim 7, wherein the piston comprises a threaded connection between a superior piston element and an inferior piston element.

15. An orthopedic implant comprising:
(a) a base section having a bone contact surface configured to be positionable in contact with a bone;
(b) a top section opposite to the base section and having a tendon contact surface configured to change a position of a patellar tendon by elevating the patellar tendon when the bone contact surface is engaged with the bone;
(c) a fixation mechanism adapted to attach the orthopedic implant to the bone; and
(d) an adjustment mechanism adapted to change a distance between the tendon contact surface and the bone contact surface at a lateral side or a medial side of the implant such that the tendon contact surface is configured to urge the patellar tendon medially or laterally, wherein the adjustment mechanism comprises a rotatable cam disposed between the top section and the base section.

16. The orthopedic implant of claim 15, wherein the patellar tendon is urged medially or laterally when the adjustment mechanism causes the change in the distance between the tendon contact surface and the bone contact surface such that the patellar tendon is also caused to rotate around its longitudinal axis.

17. The orthopedic implant of claim 16, wherein the rotation of the patellar tendon around its longitudinal axis causes rotation of the patella.

18. The orthopedic implant of claim 15, wherein the rotatable cam is disposed near the lateral side of the implant.

19. The orthopedic implant of claim 18, wherein actuation of the rotatable cam to urge the tendon contact surface away from the bone contact surface causes the patellar tendon to move medially.

20. The orthopedic implant of claim 18, wherein actuation of the rotatable cam to urge the tendon contact surface toward the bone contact surface causes the patellar tendon to move laterally.
